(19) United States
(12) Patent Application Publication
(10) Pub. No.: US 2023/0001060 A1
(43) Pub. Date: Jan. 5, 2023

(54) BREAST PUMP ADAPTER

(71) Applicant: Thistle Holdings, LLC, Chantilly, VA (US)

(72) Inventor: Brett Alan BURAN, Chantilly, VA (US)

(73) Assignee: Thistle Holdings, LLC

(21) Appl. No.: 17/363,893

(22) Filed: Jun. 30, 2021

(51) Int. Cl. A61M 1/06

(52) U.S. Cl. CPC A61M 1/062

(57) ABSTRACT

A breast pump adapter includes an upper cylindrical portion having an opening and a threading formed within a sidewall thereof. A lower cup-shaped portion extends from the upper cylindrical portion and has multiple openings. A pair of opposed flanges extend from the upper cylindrical portion and each flange has a projection formed at a distal end thereof. A lower portion is configured to be rotatably attached to the upper cylindrical portion. A guard member is disposed within an opening of the lower portion. The upper portion is configured to rotate with respect to the lower portion to move the guard member from an open position to a closed position. The breast pump adapter is configured to be attached at a first end to a breast pump and at a second end to a bottle.

### BACKGROUND OF THE INVENTION

### Field of the Invention

The present invention generally relates to breast pumps and, more particularly, to an adapter for connecting a breast pump to a milk bottle.

### Description of the Related Art

A breast pump is a device that lactating women use to extract milk from their breasts. Breasts pumps are generally mechanical devices that can be manually powered (e.g., hand powered) or automatic devices powered by electricity.

Breast pumps are used for many reasons. For example, parents use breast pumps so they can continue to breastfeed after returning to work. That is, a parent can express breast milk at work and then later bottle-feed the breast milk to their child. A breast pump may also be used to stimulate lactation for women with a low milk supply or those who have not just given birth. A breast pump may be also used to address challenges parents may encounter breast feeding, including difficulty latching, separation from an infant in intensive care, to feed an infant who cannot extract sufficient milk itself from the breast, to avoid passing medication through breast milk to the baby, or to relieve engorgement, a painful condition whereby the breasts are overfull. Pumping is often used to continue lactation and its associated hormones to aid in recovery from pregnancy even if the pumped milk is not later fed to a child.

Most breast pumps direct the pumped milk into a container (i.e., bottle) that can be used for storage and feeding. Thus, the expressed breast milk may be stored and later fed to a baby by bottle.

Conventionally, breast pumps are configured to threadably engage an opening of a bottle. Typical baby bottles have a threaded opening, which, when filled, are configured to threadably receive a milk dispensing cap or artificial nipple. Thus, in operation, after the expressed milk has been collected in the bottle, the user must disengage the breast pump from the bottle and then secure the cap to the bottle. During this exchange the bottle is left open and subject to spilling of the expressed breast milk contained in the bottle.

Furthermore, during pumping, if there is a lot of milk being expressed, there may be more milk than the bottle can hold. The pump needs to be turned off, the bottle switched or emptied, and then the pump turned back on. It can be difficult for a lactating parent to have another letdown after stopping mid pumping.

### SUMMARY OF THE INVENTION

In view of the foregoing and other exemplary problems, drawbacks, and disadvantages of the conventional methods and structures, an exemplary feature of the present invention is to provide.

In a first, exemplary, non-limiting aspect of the present invention, a breast pump adapter includes a main body and a lower portion configured to be rotatably attached to the main body. The main body includes an upper cylindrical portion, including a first end, a second end opposite to the first end, an opening formed in the first end, a cylindrical sidewall connecting the first end to the second end and a threading formed within the cylindrical sidewall, a lower cup-shaped portion extending from the second end of the upper cylindrical portion, the lower cup-shaped portion having multiple openings, and a pair of opposed flanges extending from the second end of the upper cylindrical portion, each of the flanges having a projection formed at a distal end thereof. The lower portion includes a first end, a second end opposite to the first end, an opening formed in the first end, a cylindrical sidewall connecting the first end to the second end, a lip extending around an outer circumference of the second end of the lower portion and configured to engage the projection of each of the flanges and a guard member disposed within the opening of the lower portion. The upper portion is configured to rotate with respect to the lower portion to move the guard member from an open position in which the guard member does not block the multiple openings to a closed position in which the guard member blocks the multiple openings.

In a second, exemplary, non-limiting aspect of the present invention, a breast pump assembly including a breast pump adapter and a breast pump configured to be attached to the breast bump adapter. The breast pump adapter includes a main body and a lower portion configured to be rotatably attached to the main body. The main body includes an upper cylindrical portion, including a first end, a second end opposite to the first end, an opening formed in the first end, a cylindrical sidewall connecting the first end to the second end and a threading formed within the cylindrical sidewall, a lower cup-shaped portion extending from the second end of the upper cylindrical portion, the lower cup-shaped portion having multiple openings, and a pair of opposed flanges extending from the second end of the upper cylindrical portion, each of the flanges having a projection formed at a distal end thereof. The lower portion includes a first end, a second end opposite to the first end, an opening formed in the first end, a cylindrical sidewall connecting the first end to the second end, a lip extending around an outer circumference of the second end of the lower portion and configured to engage the projection of each of the flanges and a guard member disposed within the opening of the lower portion. The upper portion is configured to rotate with respect to the lower portion to move the guard member from an open position in which the guard member does not block the multiple openings to a closed position in which the guard member blocks the multiple openings.

In a third, exemplary, non-limiting aspect of the present invention, a system includes an adapter, a breast pump configured to be attached to the breast bump adapter by threadable engagement and a bottle configured to be attached to the adapter. The breast pump adapter includes a main body and a lower portion configured to be rotatably attached to the main body. The main body includes an upper cylindrical portion, including a first end, a second end opposite to the first end, an opening formed in the first end, a cylindrical sidewall connecting the first end to the second end and a threading formed within the cylindrical sidewall, a lower cup-shaped portion extending from the second end of the upper cylindrical portion, the lower cup-shaped portion having multiple openings, and a pair of opposed flanges extending from the second end of the upper cylindrical portion, each of the flanges having a projection formed at a distal end thereof. The lower portion includes a first end, a second end opposite to the first end, an opening formed in the first end, a cylindrical sidewall connecting the first end to the second end, a lip extending around an outer circumference of the second end of the lower portion and configured to engage the projection of each of the flanges and a guard member disposed within the opening of the lower portion. The upper portion is configured to rotate with respect to the lower portion to move the guard member from an open position in which the guard member does not block the multiple openings to a closed position in which the guard member blocks the multiple openings.

In accordance with the above, exemplary aspects of the present invention, the adapter is configured to be secured at a first end to a breast pump and at a second end to a bottle. Accordingly, the openings in the adapter are configured to be arranged in an open position while the breast pump is pumping milk into the bottle. This allows milk to freely flow from the pump through the adapter and into the bottle. When the pumping is complete and the bottle is filled or partially filled, the adapter is configured to close the openings in the adapter. Thus, the opening of the bottle is closed, and milk cannot flow out of the bottle while the breast pump is removed from the bottle. Accordingly, the adapter prevents the contents of the bottle from spilling while and after the breast pump is removed from the bottle. Furthermore, the adapter will all for a bottle to be swapped while pumping without spilling milk.

### BRIEF DESCRIPTION OF THE DRAWINGS

The foregoing and other exemplary purposes, aspects and advantages will be better understood from the following detailed description of an exemplary embodiment of the invention with reference to the drawings, in which:

FIG. 1 illustrates a perspective view of a breast bump adapter 100 in accordance with an exemplary embodiment of the present invention;

FIG. 2 is an exploded perspective view of the adapter illustrated in FIG. 1;

FIG. 3 is a bottom exploded perspective view of the adapter illustrated in FIG. 1;

FIG. 4 is a cross-sectional plan view of the adapter 100 illustrated in FIG. 1;

FIG. 5 is a cross-sectional plan view of the adapter 100 illustrated in FIG. 1 connected to a bottle B;

FIG. 6 is a top perspective view of the adapter 100 illustrated in FIG. 1 rotated into a closed position;

FIG. 7 illustrates the adapter 100 connected to a breast pump P;

FIG. 8 is a bottom perspective view of the adapter 100 illustrated in FIG. 1 rotated into an open position;

FIG. 9 is a top perspective view of the adapter 100 illustrated in FIG. 1 rotated into a closed position;

FIG. 10 is an exploded top perspective view of an adapter 200 according to a second exemplary embodiment of the present invention;

FIG. 11 is an exploded perspective view of the adapter 200;

FIG. 12 is a top view of a lower portion of the adapter 200;

FIG. 13 is a bottom perspective view of the upper portion of the adapter; and

FIGS. 14 and 15 are side views of the adapter.

### DETAILED DESCRIPTION OF EXEMPLARY EMBODIMENTS OF THE INVENTION

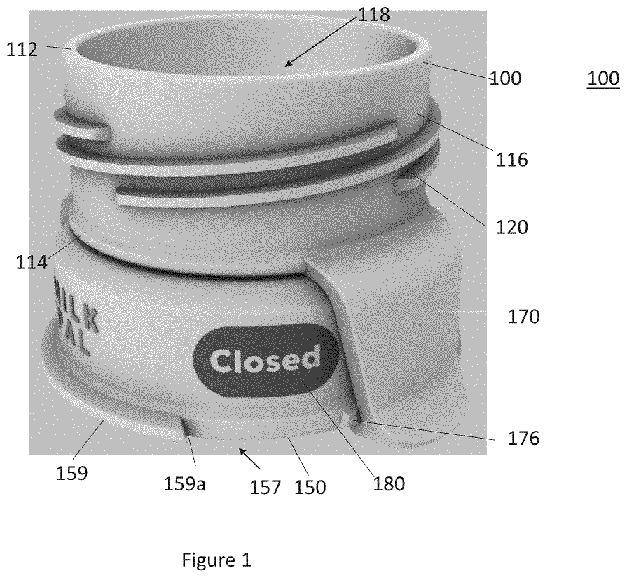

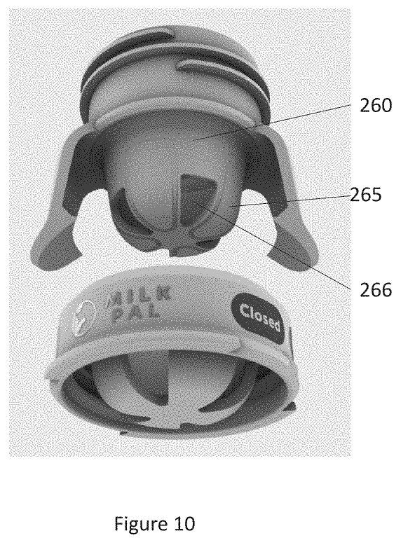

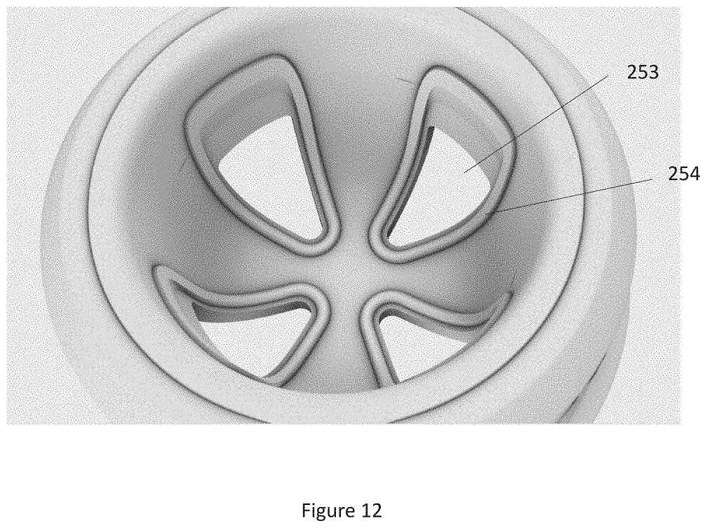

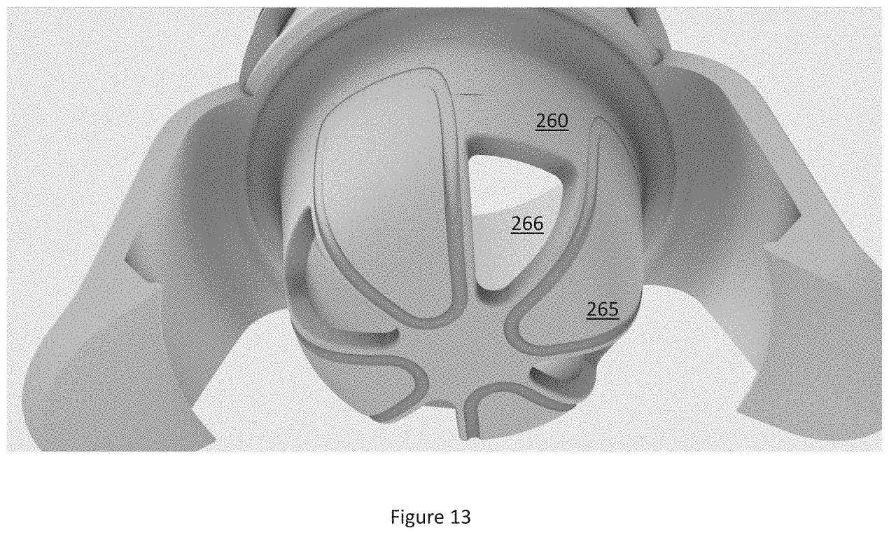

Referring now to the drawings, and more particularly to FIGS. 1-15, there are shown exemplary embodiments of the breast pump adapter and corresponding breast pump system according to the present invention.

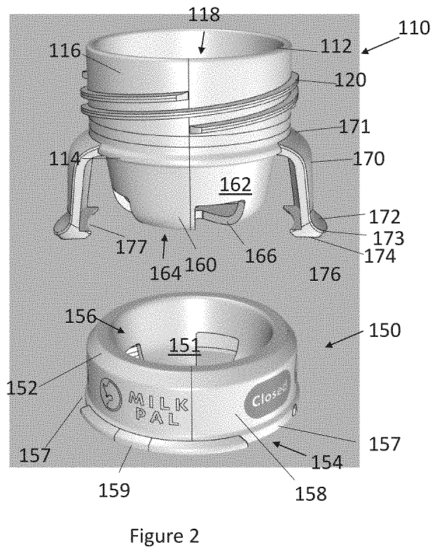

FIG. 1 illustrates a breast pump adapter 100 in accordance with a non-limiting, exemplary embodiment of the present invention. The adapter 100 includes a main body including an upper cylindrical portion 110 and a lower portion 150 configured to be rotatably attached to the upper cylindrical portion. FIG. 2 illustrates the upper cylindrical portion 110 separate from the lower portion 150.

The upper cylindrical portion 110 includes a first end 112 and a second end 114 opposite to the first end 112. A cylindrical sidewall 116 connects the first end 112 to the second end 114. An opening 118 formed in the first end 112. The opening 118 is circular and extends from the first end 112 to the second end 114, creating a cylindrical passage through the upper cylindrical portion. A threading 120 is formed within the cylindrical sidewall 116 along an outer periphery of the sidewall 116.

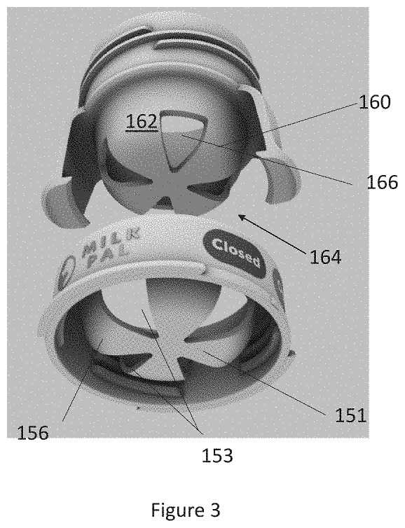

Referring to FIG. 2, a lower cup-shaped portion 160 extends from the second end 114 of the upper cylindrical portion 110. As is illustrated in FIG. 3, the lower cup-shaped portion 160 has a generally cylindrical main body 162 extending from the second end 114 to a generally flat bottom portion 164 forming a cup or bowl shape. The lower cup-shaped portion 160 has multiple openings 166 formed therein. For example, in the present embodiment, four openings are formed in the lower cup-shaped portion 160. The openings 166 are curved such that they extend along the main body portion 164 into the bottom portion 164.

A pair of opposed flanges 170 extend from the second end 114 of the upper cylindrical portion 110. The flanges 170 extend from the second end 114 in a same direction as the lower cup-shaped portion 160 having a proximal end 171 attached to the second end 114 of the upper cylindrical portion 110 and a distal end 173 opposite to the proximal end 171. The distal end 173 has a flat bottom surface 174. Each of the flanges 170 has a projection 176 and a tab 172 formed on opposite sides of the flat bottom surface 174.

The lower portion 150, as illustrated in, for example, FIG. 2, has a first end 152, a second end 154 opposite to the first end 152, an opening 156 formed in the first end 152, a cylindrical sidewall 158 connecting the first end 152 to the second end 154, and a lip 159 extending around an outer circumference of the second end 154 of the lower portion 150. A pair of cut-out portions 157 are formed within the lip 159 at opposing sides of the lower portion 150.

As is illustrated in FIGS. 2 and 3, the opening 156 extends through the entire lower portion 150. A cup-shaped guard member 151 is disposed within the opening 156 of the lower portion 150. The guard member 151 has multiple openings 153 formed therein. For example, in the present embodiment, four openings 153 are formed in the guard member 151. The openings 153 are curved such that they extend along the sides of the guard member 151 to a bottom of the guard member 151.

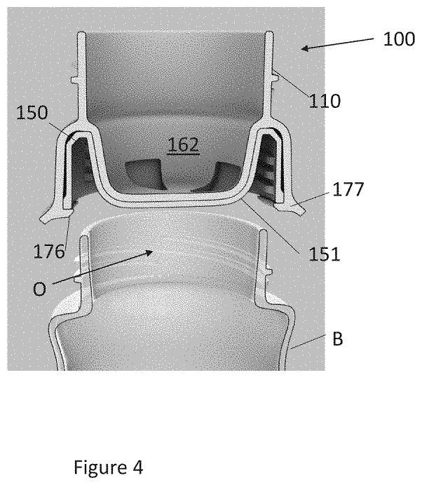

FIG. 4 illustrates a cross-sectional view of the breast pump adapter 100 positioned adjacent a bottle B. The upper cylindrical portion 110 is configured to be received in the lower portion 150. Specifically, the generally cylindrical main body 162 of the lower cup-shaped portion 160 is received through the opening 156 in the lower portion 150 and sits within the guard member 151.

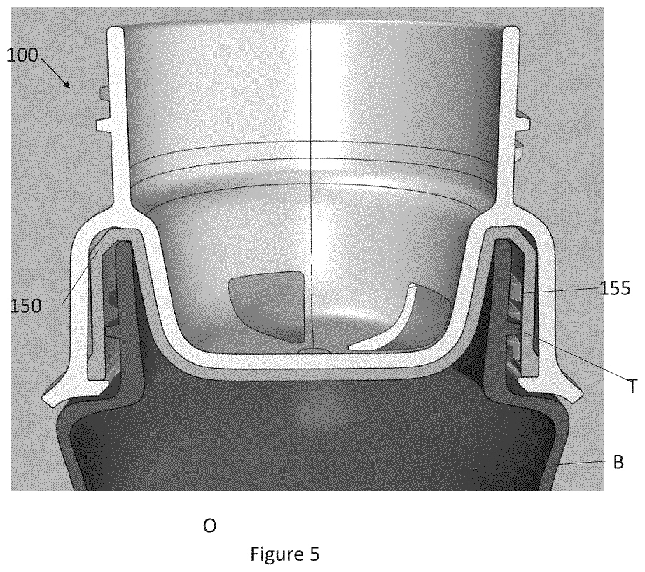

With reference to FIG. 5, the breast pump adapter 100 is configured to be removably fixed to an opening O in the bottle B. Specifically, the breast pump adapter 100 is configured to be inserted into the opening O of the bottle B. The lower portion 150 includes internal threading 155 formed on an internal wall of the lower portion 150. The internal threading 155 is configured to threadably engage external threading T on the opening O of the bottle B such that the breast pump adapter 100 is removably fixed to the opening O in the bottle B.

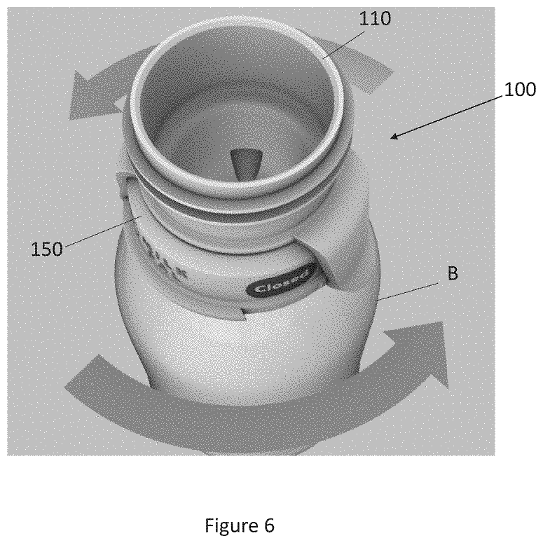

FIG. 6 illustrates the breast pump adapter 100 attached to the bottle B. When the breast pump adapter 100 is attached to the bottle B, the upper portion 110 of the breast pump adapter 100 is configured to be rotated with respect to the lower portion 150, as indicated by the arrows in FIG. 6. Specifically, referring again to FIGS. 1, 2 and 4, the projection 176 of the upper portion 176 is configured to engage with the lip 159. That is, the projection 176 attaches along a bottom of the surface of the lower portion 150, such that the bottom surface of the lower portion 150 rests on a flat top surface 177 of the projection 176 and the lower portion 150 is held between the flanges 170. The projection 176 engages the lower portion 150 within the pair of cut-out portions 157 and is movable within the pair of cut-out portions 157. End faces 159a of the lip 159 act as stoppers preventing over-rotation of the upper portion. That is, the end faces 159a prevent the projection 176 from being rotated beyond the cut-out portions 157.

The upper portion 110 is configured to rotate with respect to the lower portion 150 to move the guard member 151 from an open position in which the guard member 151 does not block the multiple openings 166 of the upper portion 110 to a closed position in which the guard member 151 blocks the multiple openings 166.

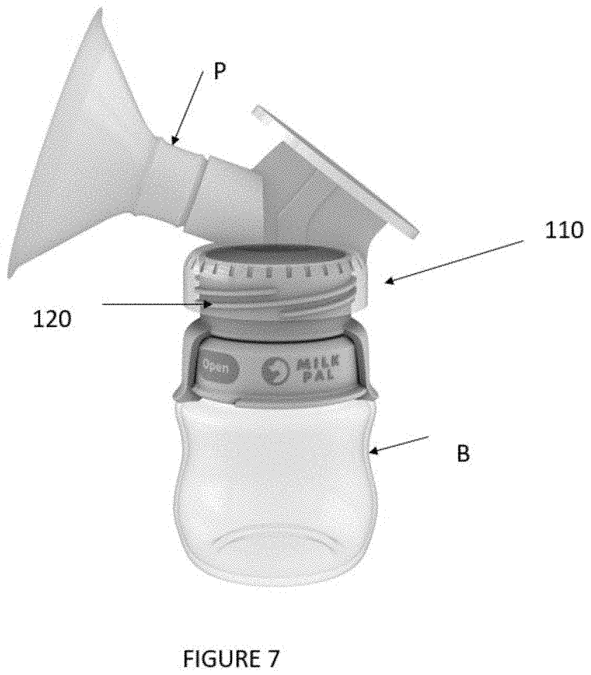

In use, the adapter 110 is configured to be secured at a first end to a breast pump P and at a second end to a bottle B. Specifically, referring to FIG. 7, the threads 120 along the upper portion 110 of the adapter 100 are configured to threadably engage corresponding threads on a breast pump P, while, as detailed above, internal threading 155 is configured to threadably engage external threading T on the opening O of the bottle B such that the breast pump adapter 100 is removably fixed to the opening O in the bottle B.

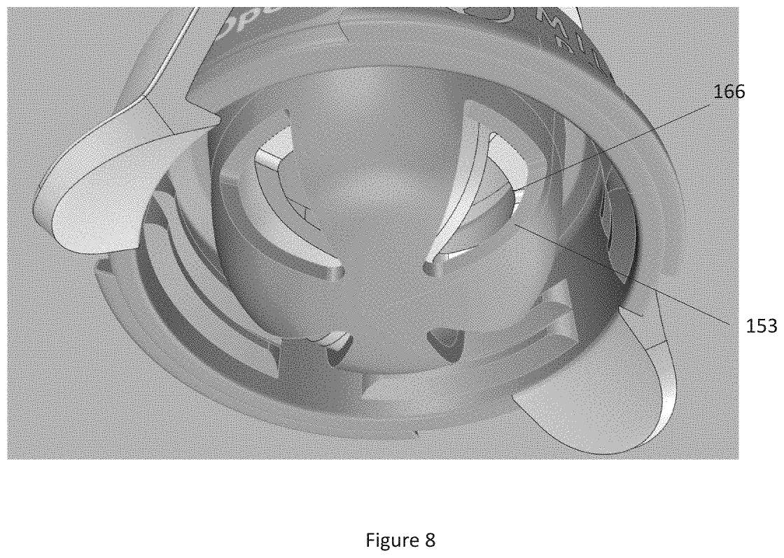

Accordingly, the openings 166 in the upper portion 110 are configured to be arranged in an open position while the breast pump P is pumping milk into the bottle B. This allows milk to freely flow from the pump through the adapter and into the bottle. The open position is illustrated in FIG. 8. That is, during pumping, the upper portion 110 is rotated to an open position in which the openings 166 of the upper portion are aligned with the openings 153 in the guard member 151. This allows milk to freely flow from the pump P, through the openings 166/151 and into the bottle B.

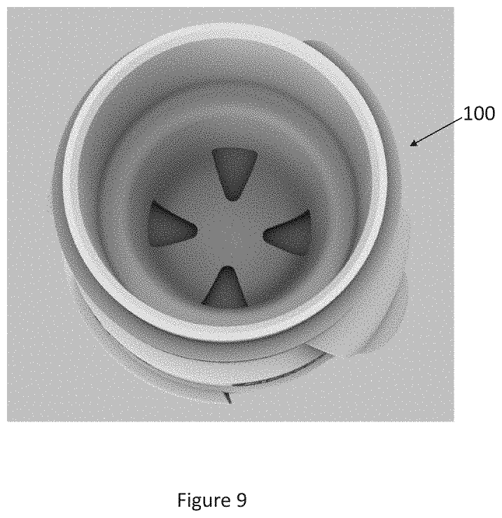

When the pumping is complete and the bottle B is filled, the adapter 100 is configured to close the openings in the adapter. FIG. 9 illustrates the adapter 100 in the closed position. In the closed position, the upper portion 110 is rotated such that the openings 166 are blocked by the guard member. Thus, the opening O of the bottle B is closed and milk cannot flow out of the bottle B while the breast pump P is removed from the bottle B. Accordingly, the adapter 100 prevents the contents of the bottle B from spilling while and after the breast pump P is removed from the bottle B.

Finally, the adapter 100 includes a status indicator 180, which indicates whether the adapter 100 is in the open or closed position. When the adapter 100 is in the closed position, the status indicator 180, which reads “Closed” will be visible, as illustrated in FIG. 1, and a corresponding status indicator 180, which reads “Open” will be blocked by one of the flanges 170. Alternatively, when the adapter 100 is in the open position, the status indicator 180, which reads “Open” will be visible (not illustrated) and a corresponding status indicator 180, which reads “Closed” will be blocked by one of the flanges 170.

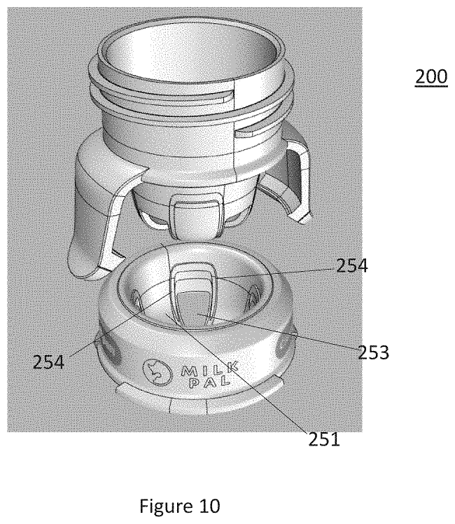

FIGS. 10-13 illustrate a second exemplary embodiment of the present invention. The adapter 200 of the second exemplary embodiment is substantially identical to the adapter 100 detailed above, with the additional feature of beading to improve the seal within the adapter 200.

Specifically, the multiple holes 253 formed in the guard member 251 have a beading 254 formed around the holes along an interior of the guard member 251. Similarly, the upper portion 260 the adapter 200 has beadings 265 formed on an outer surface of the upper portion 260 between the multiple openings 266. The beadings 265 are projections formed on the outer surface of the upper portion 260, which are configured to be received in the beadings 254 of the guard member 251 when the adapter 200 is in the closed position. The beadings 254/266 further seal the holes 253/266 when the adapter 200 is in the closed position.

With respect to each of the embodiments detailed above, the adapter is preferably made from a food safe plastic. Additionally, the entire upper portion 110, including the flanges 170, may be formed as a single, integral piece. Similarly, the entire bottom portion 150, including the guard member 151, may be formed as a single, integral piece.

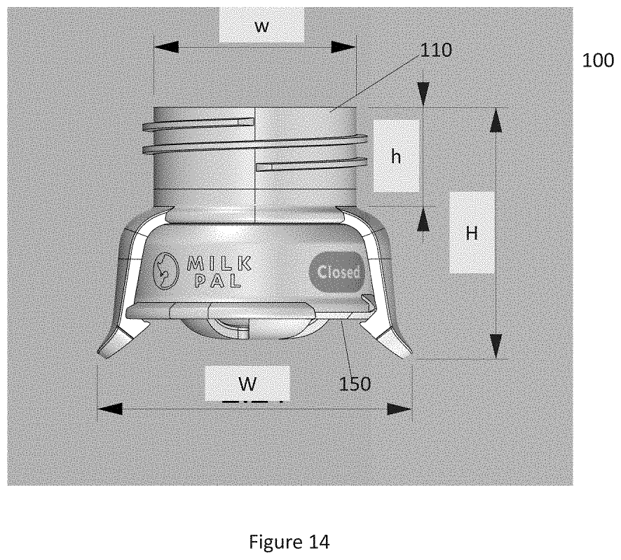

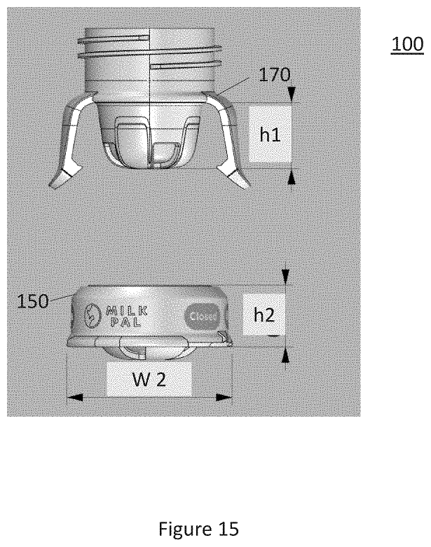

The adapter may be formed in varying sizes designed to fit specific breast pumps and bottles on the market. With reference to FIGS. 14 and 15, the adapter may have an overall width (i.e., the width measured from the furthest points of separate at the tabs 172) W in a range of 2.21 in-2.76 in. Specific embodiments of the adapter have a width W of 2.21 in, 2.69 or 2.76 in. Additionally, the adapter may have an overall, assembled height H of 1.76 in-1.92 in. Specific embodiments of the adapter have a height H of 1.76 in, 1.90 or 1.92 in. The opening 118 in the upper portion 110 has a width win a range of 1.42 in-1.95 in. Specific embodiments of the adapter having an opening 118 with a width w of 1.42 in or 1.95 in. The upper portion 110 may have a height h in a range of 0.70 in-0.86 in. Specific embodiments of the adapter have an upper portion 110 with a height h of 0.70 in, 0.84 in or 0.86 in. The flange 170 may have a height h1 that 0.72 in-0.73 in. The lower portion 150 may have a height h2 in a range of 0.66 in-0.80 in. Specific embodiments of the adapter have a lower portion 150 with a height h2 of 0.66 in, 0.67 in or 0.80 in. The lower portion 150 may have a width w2 in a range of 1.80 in-2.32 in. Specific embodiments of the adapter have a lower portion 150 with a width w2 of 1.80 in, 1.81 in or 2.32 in.

While the invention has been described in terms of several exemplary embodiments, those skilled in the art will recognize that the invention can be practiced with modification within the spirit and scope of the appended claims.

Further, it is noted that, Applicant's intent is to encompass equivalents of all claim elements, even if amended later during prosecution.

What is claimed is:

1. A breast pump adapter, comprising:
a main body, comprising:
an upper cylindrical portion, comprising:
a first end;
a second end opposite to the first end;
an opening formed in the first end;
a cylindrical sidewall connecting the first end to the second end; and
a threading formed within the cylindrical sidewall;
a lower cup-shaped portion extending from the second end of the upper cylindrical portion, the lower cup-shaped portion having multiple openings; and
a pair of opposed flanges extending the second end of the upper cylindrical portion, each of the flanges having a projection formed at a distal end thereof; and
a lower portion configured to be rotatably attached to the upper cylindrical portion, the lower portion comprising:
a first end;
a second end opposite to the first end;
an opening formed in the first end;
a cylindrical sidewall connecting the first end to the second end;
a lip extending around an outer circumference of the second end of the lower portion and configured to engage the projection of each of the flanges; and
a guard member disposed within the opening of the lower portion,
wherein the upper portion is configured to rotate with respect to the lower portion to move the guard member from an open position in which the guard member does not block the multiple openings to a closed position in which the guard member blocks the multiple openings.

2. The breast pump adapter according to claim 1, further comprising a pair of cut-out portions formed within the lip.

3. The breast pump adapter according to claim 1, further comprising a stopper configured to limit rotation of the upper cylindrical portion.

4. The breast pump adapter according to claim 1,

5. The breast pump adapter according to claim 1, wherein the guard member comprises multiple openings.

6. The breast pump adapter according to claim 5, further comprising a first beading formed in the multiple openings of the guard member.

7. The breast pump adapter according to claim 6, further comprising a second beading formed between the multiple openings in the lower-cup shaped portion configured to be received in the first beading.

8. The breast pump adapter according to claim 1, wherein main body is a single integral piece.

9. The breast pump adapter according to claim 1, wherein the lower portion is a single integral piece.

10. The breast pump adapter according to claim 1, wherein the main body and the lower portion are made of food-safe plastic.

11. A breast pump assembly, comprising:
a breast pump adapter, comprising:
a main body, comprising:
an upper cylindrical portion, comprising:
a first end;
a second end opposite to the first end;
an opening formed in the first end;
a cylindrical sidewall connecting the first end to the second end; and
a threading formed within the cylindrical sidewall;
a lower cup-shaped portion extending from the second end of the upper cylindrical portion, the lower cup-shaped portion having multiple openings; and
a pair of opposed flanges extending the second end of the upper cylindrical portion, each of the flanges having a projection formed at a distal end thereof; and
a lower portion configured to be rotatably attached to the upper cylindrical portion, the lower portion comprising:
a first end;
a second end opposite to the first end;
an opening formed in the first end;
a cylindrical sidewall connecting the first end to the second end;
a lip extending around an outer circumference of the second end of the lower portion and configured to engage the projection of each of the flanges; and
a guard member disposed within the opening of the lower portion; and
a breast pump configured to be attached to the breast bump adapter by threadable engagement with the threading,
wherein the upper portion is configured to rotate with respect to the lower portion to move the guard member from an open position in which the guard member does not block the multiple openings to a closed position in which the guard member blocks the multiple openings.

12. A system comprising:
an adapter, comprising:
a main body, comprising:
an upper cylindrical portion, comprising:
a first end;
a second end opposite to the first end;
an opening formed in the first end;
a cylindrical sidewall connecting the first end to the second end; and
a threading formed within the cylindrical sidewall;
a lower cup-shaped portion extending from the second end of the upper cylindrical portion, the lower cup-shaped portion having multiple openings; and
a pair of opposed flanges extending the second end of the upper cylindrical portion, each of the flanges having a projection formed at a distal end thereof; and
a lower portion configured to be rotatably attached to the upper cylindrical portion, the lower portion comprising:
a first end;
a second end opposite to the first end;
an opening formed in the first end;
a cylindrical sidewall connecting the first end to the second end;
a lip extending around an outer circumference of the second end of the lower portion and configured to engage the projection of each of the flanges; and
a guard member disposed within the opening of the lower portion;
a breast pump configured to be attached to the breast bump adapter by threadable engagement with the threading; and
a bottle configured to be attached to the lower portion of the adapter,
wherein the upper portion is configured to rotate with respect to the lower portion to move the guard member from an open position in which the guard member does not block the multiple openings to a closed position in which the guard member blocks the multiple openings.
